(19) United States
(12) Patent Application Publication
(10) Pub. No.: US 2006/0237021 A1
(43) Pub. Date: Oct. 26, 2006

(54) Methods and devices for rejuvenating skin

(76) Inventors: Gordon G. Guay, Chelmsford, MA (US); Natalia Botchkareva, Sharon, MA (US); Gurpreet S. Ahluwalia, Wellesley, MA (US); Douglas Shander, Acton, MA (US)

Correspondence Address: FISH & RICHARDSON P.C., P.O. BOX 1022, MINNEAPOLIS, MN, 55440-1022, US

(21) Appl. No.: 11/111,564

(22) Filed: Apr. 21, 2005

(51) Int. Cl. A61B 19/00

(52) U.S. Cl. 128/898

(57) ABSTRACT

Methods and devices are provided for rejuvenating the skin of a subject, e.g., a human. The methods include moving a blade across a skin region of the subject. The skin region, for example, can include predominantly vellus hairs.

### TECHNICAL FIELD

This invention relates to methods and devices for rejuvenating skin.

### BACKGROUND

Skin care can be of particular importance in improving or enhancing the appearance of men and women. Various products and methods can be used to care for skin. For example, exfoliant scrubs, cleansers, and lotions are sometimes used to maintain healthy-looking skin. Exfoliant scrubs can be used to remove dead skin cells from the surface of the skin, which can give the skin an improved tone. Soaps and other cleansers can be used to remove dirt and excess oil from the skin, which can help prevent clogging of pores. Consequently, acne and other types of skin blemishes can be prevented in some cases. Lotions and various other topical ointments can also be used to deliver nutrients and/or moisturizers to the skin in an effort to improve the appearance and/or the health of the skin. Other types of cosmetic products (e.g., creams and lotions) or drug actives are sometimes used in an attempt to eliminate wrinkling and other signs of aging.

### SUMMARY

The invention features methods and devices for rejuvenating skin. As used herein, skin rejuvenation includes one or more of the following: reducing the appearance of fine lines and wrinkles; reducing deep wrinkles; enhancing skin tone and elasticity; reducing skin blemishes and/or age spots; reducing skin roughness; and producing a younger looking skin.

In one aspect, the invention features a method of rejuvenating skin of a subject, which includes moving at least one blade across a region of the skin having predominantly vellus hairs thereon.

Implementations can include one or more of the following features.

In certain implementations, the method includes moving at least one blade across the skin of a periorbital of the subject.

In some implementations, the method includes moving at least one blade across the skin of a forehead of the subject.

In certain implementations, the method includes moving at least one blade across the skin of a cheek of the subject.

In some implementations, moving the at least one blade causes the skin to release one or more types of cytokines.

In certain implementations, the method further includes applying a treatment to the skin.

In some implementations, the applying step includes exfoliation.

In certain implementations, the applying step includes mechanical exfoliation.

In some implementations, the applying step includes delivering energy (e.g, thermal energy, photothermal energy, IR energy, and/or visible light) to the skin.

In certain implementations, the treatment is applied prior to moving the blade across the skin.

In some implementations, the treatment is applied while moving the blade across the skin.

In certain implementations, the treatment is applied after moving the blade across the skin.

In some implementations, no greater than about ten percent of the hair on the region is terminal hair.

In certain implementations, the method includes moving multiple blades across the skin.

In some implementations, the blades have different relative sharpnesses.

In certain implementations, the blades are oriented at different angles.

In some implementations, the method further includes applying a skin-rejuvenating product to the skin.

In certain implementations, the skin rejuvenating product includes a moisturizer, a sun screen product, an anti-acne product, an anti-wrinkle product, a depigmentation product, a product for increasing skin turn-over, tretinoin, vitamins, and/or anti-irritants.

In some implementations, the skin-rejuvenating product is applied after moving the blade across the skin.

In certain implementations, the skin-rejuvenating product is applied while moving the blade across the skin.

In some implementations, the skin-rejuvenating product is applied prior to moving the blade across the skin.

The methods can include one or more of the following advantages.

In certain implementations, skin can be rejuvenated without the need for expensive cosmetic products. In some implementations, the skin can be rejuvenated without perceived skin irritation. In certain implementations, the skin can be rejuvenated using a simple technique that can easily be incorporated into the user's regular hygiene regimen. In some implementations, the user can rejuvenate and/or exfoliate skin in a controlled fashion, which can reduce the amount of irritation to the user's skin.

Other features and advantages of the invention are in the description, the drawings, and the claims.

### DESCRIPTION OF DRAWINGS

FIG. 1 is a cross-sectional view of an implementation of a skin-rejuvenating device.

FIG. 2 is a bottom view of the implementations of the skin-rejuvenating device of FIG. 1.

FIG. 3 is an enlarged view of region A of FIG. 1.

### DETAILED DESCRIPTION

Generally, the skin-rejuvenation methods discussed herein include moving a skin-rejuvenating device across a region of skin. The skin-rejuvenating device includes one or more sharpened blades positioned to contact the skin surface as the skin-rejuvenating device is moved across the skin region. Consequently, the blade(s) remove live skin cells from the skin region, which, as discussed below, can help rejuvenate the skin.

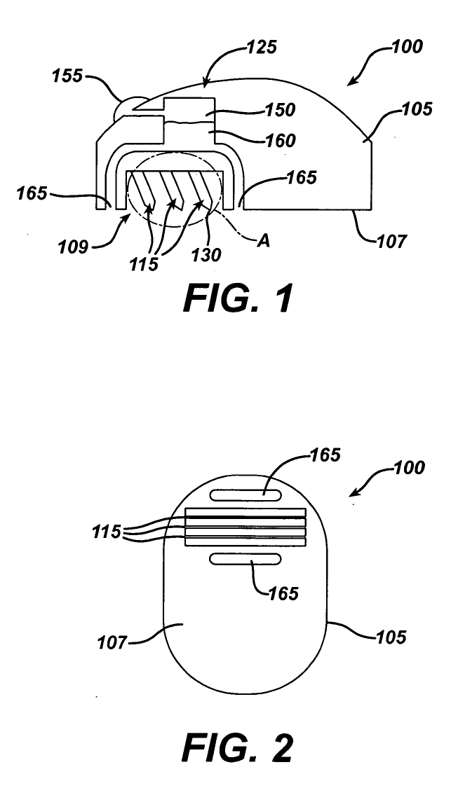

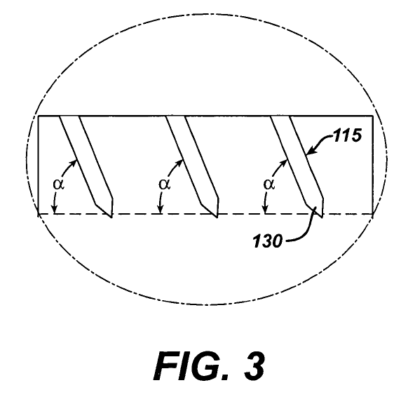

Referring to FIGS. 1 and 2, a skin-rejuvenating device 100 includes a housing 105 having a bottom surface 107. Housing 105 defines a recess 109 extending inwardly from bottom surface 107. Blades 115 are retained within recess 109, and extend transversely across the housing. Each of blades 115 includes a sharpened edge 130 directed generally toward bottom surface 107. As shown in FIG. 3, blades 115 are arranged at an angle of about 15 degrees to about 30 degrees (e.g., about 20 degrees to about 25 degrees) relative to the bottom surface 107. As discussed in more detail below, this orientation of blades 115 can cause sharpened edges 130 of blades 115 to create minor trauma or damage to the user's skin surface when skin-rejuvenating device 100 is moved across the user's skin. The blade sharpness can be adjusted to increase the comfort of the user during movement of skin-rejuvenating device 100 across the skin surface. Alternatively or additionally, sharpened edges 130 can be coated (e.g., with a Telomeric coating) to increase the comfort of the user during as skin-rejuvenating device 100 is moved across the user's skin.

As shown in FIG. 1, housing 105 also includes a skin care product delivery mechanism 125. Delivery mechanism 125 includes a storage compartment 150, a skin care product 160 contained within storage compartment 150, an activator button 155, and outlets 165. Outlets 165 are defined by bottom surface 107 of housing 105, and are located adjacent opposing ends of recess 109. Outlets 165 are in fluid communication with storage compartment 150. For example, tubing may be used to connect storage compartment 150 to outlets 165.

Activator button 155 is configured to activate a valve upon being pressed, which can pressurize storage compartment 150. Upon pressing button 155 one or more times, the pressure within storage compartment 150 can increase, causing skin care product 160 to be forced through outlets 165. In some implementations, skin care product 160 includes an exfoliator. For example, skin care product can include a chemical exfoliator, such as alpha hydroxy acids and/or beta hydroxy acids. As another example, skin care product 160 can include a mechanical exfoliator, such as aluminum oxide. Any of various other mechanical exfoliators can alternatively or additionally be used. The mechanical exfoliator(s) can include particles of any of various shapes and sizes. The particles can, for example, be any of various three-dimensional shapes that include relatively discrete edges. While skin care product 160 has been described as an exfoliator, skin care product 160 can alternatively or additionally be a moisturizer, a sunscreen product, an anti-acne product, an anti-wrinkle product, a depigmenting product, a product for increasing skin turnover, tretinoin, vitamins, and/or anti-irritants.

A method of using skin-rejuvenating device 100 involves first selecting a region of skin to rejuvenate. After selecting the region of skin to rejuvenate, the user scrapes blades 115 of skin-rejuvenating device 100 across the selected region and applies treatment (e.g., skin care product) to the selected region. This process can help rejuvenate the selected region of skin.

Selecting the region of skin to rejuvenate includes selecting any of various skin regions from which predominantly vellus hairs (e.g., relatively fine, soft, and non-pigmented hairs) extend. In some implementations, about ten percent or less (e.g., about five percent to about ten percent) of the hair on the selected region is terminal hair (e.g., relatively long, coarse, and pigmented hair). In certain implementations, vellus hairs account for about 90 percent or more (e.g., about 95 percent or more) of the hair on the selected region or regions. Skin regions with predominantly vellus hairs can include any of various facial regions, such as the periorbital region, the upper lip, the cheek, the chin, the forehead, and/or the neck. For many women, in particular, the selected region could be substantially any region of the face and neck. The selected skin region can alternatively or additionally be any of various other skin regions, such as regions of the hands and/or feet. The skin region selected by the user is typically a region of skin that is not regularly shaved with conventional shaving devices, such as manual or electric razors.

After selecting the region of skin to rejuvenate, the user moves skin-rejuvenating device 100 across the selected skin region with at least a portion of bottom surface 107 contacting the selected skin region. Consequently, sharpened edges 130 of blades 115 are moved across the user's skin. Blades 115 can be arranged at an angle relative to the skin surface that can allow blades 115 to cause substantially only superficial damage to the skin surface as it is moved across the skin. While being moved across the skin region, for example, blades 115 can be positioned at an angle of about 15 degrees to about 30 degrees (e.g., about 20 degrees to about 25 degrees) relative to the surface of the selected skin region. Due to the sharpness and orientation of blades 115, skin cells (e.g., live and dead skin cells) are removed as blades 115 are scraped across the user's skin surface.

Skin cells can, for example, be removed from the skin surface due to unevenness of the skin surface. The stratum corneum, for example, has many valleys and ridges that make the skin surface uneven on a microscopic level. As skin-rejuvenating device 100 is moved across the skin surface, it can cause micro-abrasions, which can result in the removal of skin cells. Generally, only a few layers (e.g., about three layers or less) of live and/or dead skin are effected (e.g., removed) by a single stroke of skin-rejuvenating device 100. In some implementations, it can be beneficial to apply about five or more (e.g., about five to about ten) strokes to remove as many as about ten layers (e.g., about five to about eight layers) of the stratum corneum in the selected region. The removed layers of skin can, for example, account for up to about ten percent (e.g., about five percent to about eight percent) of the epidermis in the selected region. As described below, it is believed that the removal of celluar debris (e.g., live and dead skin cells) can significantly increase the rate of release of cytokines from the selected skin region, which can provide a rejuvenating effect.

Live skin cells can make up about seven percent or more (e.g., about ten percent or more, about 15 percent or more, about 20 percent or more, about seven percent to about 20 percent, about seven percent to about 15 percent) of the cellular debris removed from the skin region. Without wishing to be bound by theory, it is believed that the minor perturbation (e.g., perturbation that can be transiently or rapidly reversed) caused to the selected region of skin by blades 115 (e.g., by the removal of live skin cells) initiates a wound healing cascade within the skin. The wound healing cascade can cause the selected region of skin to release one or more types of cytokines (e.g, interleukins) at an increased rate. The increased rate of release of cytokines can induce new cell turnover and proliferation, and can increase blood flow to the selected region of skin. In addition, it is believed that live cell removal can increase synthesis of growth factors and biologically active peptides, which can activate synthesis of structural proteins of the skin and further contribute to rejuvenation of the skin. As a result of the increased rate of release of cytokines and/or the increased synthesis of growth factors and biologically active peptides, the texture, firmness, and/or tone of the skin can be improved.

Generally, skin-rejuvenation levels increase as the release rate of cytokines increases, and skin-rejuvenation levels decrease as the release rate of cytokines decreases. As noted above, by using the methods described herein, the user of skin-rejuvenating device 100 can achieve an increased rate of release of cytokines as compared to a control skin site to which no such methods are applied. In some implementations, cytokines can be released from the selected skin region at a rate of about 1.3 fold or more (e.g., about, 1.5 fold or more, about 2 fold or more, about 4fold or more, about 2 fold to about 4 fold) relative to rates within the control skin site.

Due in part to the orientation of blades 115 relative to the skin surface, cellular debris (e.g., live and dead skin cells) can generally be removed from the selected region without perceived irritation to the skin. For example, substantially no redness, stinging, burning, or discomfort may occur within the selected region of skin as a result of moving skin-rejuvenating device 100 across the selected region in accordance with the description above.

While scraping blades 115 across the selected skin region, the user can additionally provide treatment to the skin. For example, the user can deliver skin care product 160 to the selected skin region by pressing activator button 155 in order to increase the pressure within cavity 150. As described above, as the pressure within cavity 150 increases, some of skin care product 160 is forced out of outlet 165 and onto the skin surface of the user. In certain implementations, as discussed above, skin care product 160 is a chemical exfoliator. In such implementations, upon applying the chemical exfoliator to the skin, extracellular matrices can be dissolved to slough off dead cells within the stratum corenum of the selected region of skin.

Using the techniques described herein, a region of skin can be rejuvenated by mild stimulation without causing substantial irritation to the skin. For example, blades 115 can create minor trauma or other types of damage in the skin surface to initiate a wound response cascade. The minor abrasions created by blades 115 can cause substantially no perceived irritation to the selected region of skin. One or more additional treatments can also be applied to the skin to enhance rejuvenation of the skin. Due to the minimal irritation created by the methods described herein, these methods can be repeated relatively frequently. These methods can, for example, be performed at least three times per week (e.g., at least five times per week, at least once per day).

While many implementations have been described above, other implementations are possible.

In some implementations, blades 115 extend at multiple different angles. In certain implementations, for example, the angle at which the blades are arranged increases from the frontmost blade to the rearmost blade. In other implementations, the angle at which the blades are arranged decreases from the frontmost blade to the rearmost blade.

In certain implementations, blades 115 have different levels of sharpness. For example, blade sharpness can increase or decrease from the frontmost blade to the rearmost blade.

In some implementations, skin rejuvenation device 100 includes only one blade 115.

In certain implementations, delivery device 125 is configured to release skin care product 160 in response to electric activation. For example, an electric actuator or pump and a power source (e.g., a battery) can be contained within housing 105. Activator button 155 can be configured to create an electrical connection between the actuator/pump upon being depressed. Thus, upon depressing activator button 155, the actuator/pump can force some of skin care product 160 out of outlet 165 and onto the surface of the user's skin.

In some implementations, delivery device 125 can be configured to release skin rejuvenation ingredient solution 160 in response to motion. For example, the delivery device 125 can have an electrically driven delivery pump that is activated upon touching skin-rejuvenating device 100 to the skin of the user to complete an open circuit. Thus, skin care product 160 can be automatically delivered to the skin upon touching the skin-rejuvenation device to the skin.

As an alternative to or in addition to exfoliator delivery device 125, other means for delivering skin care product 160 to the skin region can be used. In certain implementations, for example, skin care product 160 is included within a dissolvable matrix attached to housing 105 (e.g., attached to bottom surface 107). The dissolvable matrix can be constructed to partially dissolve when exposed to liquids, such as water. Thus, upon exposure to water, a predetermined amount of the skin care product can be released. The dissolvable matrix can be formed of any of various materials, such as a water soluble polymer systems (e.g., polyox and/or polyethylene glycol). Skin care product 160 can be positioned within the matrix using any of various standard compounding methods.

In certain implementations, skin rejuvenating device 100 includes a mechanical exfoliator. For example, bottom surface 107 of skin-rejuvenating device 100 can include a textured surface that removes live and/or dead skin cells as it is rubbed across the skin. The textured surface can include, for example, ribs, spikes, and/or other types of protrusions capable of removing skin cells as the skin-rejuvenating device is scraped across a region of the user's skin. Other examples of exfoliators are described in U.S. Published Pat. App. No. US-2004-0181943-A1 and in commonly owned and co-pending U.S. patent application Ser. No. 10/732,555, filed on Dec. 10, 2003, which are incorporated herein by reference.

In certain implementations, skin-rejuvenating device 100 does not include a skin care product delivery mechanism. In such implementations, skin care product 160 can, if desired, be delivered to the selected region of skin using other means. For example, a delivery mechanism can be included within a separate device. Thus, skin rejuvenating device 100 can be moved across the skin region before or after the skin care product 160 is delivered to the selected skin region using the separate device. In some implementations, skin care product 160 is a lotion, cream, or solution that can be applied by the hand, rather than being delivered by a delivery mechanism. For example, the user can rub the skin care product into the selected skin region with his or her fingers before or after moving skin rejuvenating device 100 across the skin region.

As an alternative to or in addition to applying skin care product 160 to the skin, other forms of treatment can be applied. In some implementations, for example, certain types of energy can be applied to the selected skin region to enhance rejuvenation. Examples of suitable types of energy include thermal energy, photothermal energy, IR energy, and visible light. Applying a low level amount of energy to the skin can produce a transient wound in the skin, which can enhance or elicit a cytokine release. In some implementations, the energy has a wavelength of about 400 nm to about 1300 nm. In certain implementations, the energy can be transmitted at an intensity of about 1.0 J/cm² to about 100 J/cm² (e.g., about 2.0 J/cm² to about 15 J/cm²). In order to provide energy delivering capability, housing 105 of skin-rejuvenating device 100, for example, can include a transmitter configured to transmit one or more of the above-noted energies to the selected region of the user's skin upon depression of activator button 155.

In some implementations, the process can include one or more steps in addition to those steps described above. For example, after moving blades 115 across the selected skin region and applying skin care product 160 and/or energy to the selected region, the user can additionally apply any of various other skin-rejuvenating products to the selected skin region. For example, the user can apply a moisturizer, a sunscreen product, an anti-acne product, an anti-wrinkle product, a depigmenting product, a product for increasing skin turnover, tretinoin, vitamins, and/or anti-irritants. These treatment products can help sooth, protect, and/or further contribute to rejuvenation of the skin.

In certain implementations, the process includes only a subset of the steps described herein. For example, the process can include only the step of scraping the blade across the skin region.

Other implementations are within the scope of the claims.

What is claimed is:

1. A method of rejuvenating skin of a subject, the method comprising:
moving at least one blade across a region of the skin having predominantly vellus hairs thereon.

2. A method of rejuvenating skin of a subject, the method comprising:
moving at least one blade across the skin of a periorbital of the subject.

3. A method of rejuvenating skin of a subject, the method comprising:
moving at least one blade across the skin of a forehead of the subject.

4. A method of rejuvenating skin of a subject, the method comprising:
moving at least one blade across the skin of a cheek of the subject.

5. The method of claim 1, 2, 3, or 4 wherein moving the at least one blade causes the skin to release one or more types of cytokines.

6. The method of claim 1, 2, 3, or 4 further comprising applying a treatment to the skin.

7. The method of claim 6 wherein the applying step comprises exfoliation.

8. The method of claim 7 wherein the applying step comprises mechanical exfoliation.

9. The method of claim 6 wherein the applying step comprises delivering energy to the skin.

10. The method of claim 9 wherein the energy comprises one or members selected from the group consisting of thermal energy, photothermal energy, IR energy, and visible light.

11. The method of claim 6 wherein the treatment is applied prior to moving the blade across the skin.

12. The method of claim 6 wherein the treatment is applied while moving the blade across the skin.

13. The method of claim 6 wherein the treatment is applied after moving the blade across the skin.

14. The method of claim 1 wherein no greater than about ten percent of the hair on the region is terminal hair.

15. The method of claim 1, 2, 3, or 4 wherein the method includes moving multiple blades across the skin.

16. The method of claim 15 wherein the blades have different relative sharpnesses.

17. The method of claim 15 wherein the blades are oriented at different angles.

18. The method of claim 1, 2, 3, or 4 further comprising applying a skin-rejuvenating product to the skin.

19. The method of claim 18 wherein the skin rejuvenating product comprises one or more members selected from the group consisting of a moisturizer, a sun screen product, an anti-acne product, an anti-wrinkle product, a depigmentation product, a product for increasing skin turn-over, tretinoin, vitamins, and anti-irritants.

20. The method of claim 18, wherein the skin-rejuvenating product is applied after moving the blade across the skin.

21. The method of claim 18, wherein the skin-rejuvenating product is applied while moving the blade across the skin.

22. The method of claim 18, wherein the skin-rejuvenating product is applied prior to moving the blade across the skin.
